(19) United States
(12) Patent Application Publication
(10) Pub. No.: US 2023/0288413 A1
(43) Pub. Date: Sep. 14, 2023

(54) LATERAL FLOW PLATFORM FOR DETECTION OF DIAGNOSTIC MARKERS

(71) Applicant: Universal Sequencing Technology Corporation, Canton, MA (US)

(72) Inventors: Peiming ZHANG, Gilbert, AZ (US); Sanjay B. HARI, Canton, MA (US); Xinyue ZHANG, Canton, MA (US); Barrett DUAN, Canton, MA (US); Ming LEI, Sharon, MA (US)

(73) Assignee: Universal Sequencing Technology Corporation, Canton, MA (US)

(21) Appl. No.: 18/299,528

(22) Filed: Apr. 12, 2023

Related U.S. Application Data
(63) Continuation of application No. PCT/US2021/055101, filed on Oct. 14, 2021.
(60) Provisional application No. 63/091,790, filed on Oct. 14, 2020.

(51) Int. Cl. G01N 33/543; G01N 21/78; B01L 3/00

(52) U.S. Cl. CPC G01N 33/54388; G01N 21/78; B01L 3/5023; G01N 2021/7759

(57) ABSTRACT

This disclosure provides, in one aspect, a platform and related methods to detect diagnostic markers or biomarkers for various diseases. In some embodiments, the platform can be a lateral flow test strip having a hydrogel composition deposited on one or more test zones.

### CROSS REFERENCE TO RELATED APPLICATIONS

This application is a U.S. utility application under 35 U.S.C. § 111(a) that is a continuation of PCT International Patent Application No. PCT/US2021/055101, filed Oct. 14, 2021, designating the United States and published in English, which claims priority to and the benefit of U.S. Provisional Application No. 63/091,790 filed Oct. 14, 2020, the entire disclosure of each of which is incorporated herein by reference.

### SEQUENCE LISTING

The present application contains a Sequence Listing which has been submitted electronically in XML format following conversion from the originally filed TXT format. The content of the electronic XML Sequence Listing, (Date of creation: Apr. 12, 2023; Size: 4,856 bytes; Name: 168460-012202US-Sequence_Listing.xml), and the original TXT format, is herein incorporated by reference in its entirety.

### FIELD

The present disclosure in general relates to methods and compositions for the detection of pathogenic markers.

### BACKGROUND

On Dec. 31, 2019, the first cases of a novel coronavirus were identified in Wuhan City, Hubei Province, China. Since then, the coronavirus disease 2019 (as known as COVID-19) has evolved into a pandemic. As of Oct. 10, 2020, there are 37,050,406 COVID-19 cases and 1,070,393 deaths reported worldwide, according to COVID-19 Dashboard by the Center for Systems Science and Engineering (CSSE) at Johns Hopkins University. There were 7,706,256 COVID-19 cases (>20% of global cases) and 214,286 deaths in the United States alone.

With the pandemic fizzled out, recurrence of SARS-CoV-2 is projected,¹ and a new strategy of testing has been called: shifting from diagnosing those who have symptoms or are exposed to the virus toward screening whole populations using faster and cheaper tests.²

Although the etiological diagnosis of SARS-CoV-2 infection has virtually been made by detecting viral RNA in biological samples, especially those extracted from the upper and lower respiratory tracts, serological testing is exploited to recognize the presence of antibodies generated through humoral response against the virus as well as neutralizing antibodies elicited by vaccine. It can monitor seroprevalence and herd immunity for epidemiology watch the nature and duration of humoral immunity, and complement nucleic acid testing for diagnosis.³ Besides, it can be employed in antibody-based therapeutics (i.e., convalescent serum or monoclonal antibodies) and vaccines (i.e., selecting non-immunized individuals and follow-up).⁴

Lateral flow immunoassays (LFIA) have been utilized for testing anti-SARS-CoV-2 antibodies due to their quickness and cheapness. During the COVID-19 pandemic, many companies have had Emergency Use Authorization of LFIAs for the SARS-CoV-2 test. However, those LFIAs are limited by their low specificity and sensitivity. Ye and coworkers have shown an LFIA test of IgG and IgM with a sensitivity of ˜89% and specificity of ˜90% from clinical samples.⁵ Also, the products from different vendors have shown varied specificities and sensitivity.⁶

Thus, improved lateral flow strips are needed.

### BRIEF DESCRIPTION OF THE DRAWINGS

FIG. 1 illustrates an exemplary lateral flow strip patterned with hydrogel detection lines.

FIG. 2 illustrates an exemplary photochemical process to generate a polyacrylamide hydrogel network with protein trapped in the patterned areas on a nitrocellulose membrane.

FIG. 3: Chromatographic lateral flow Images showing limits of detection of test strips with anchor protein molecules immobilized differently in the nitrocellulose membrane, (panel a) by conventional physical adsorption, (panel b) by hydrogel, (panel c) by hydrogel and streptavidin, and (panel d) by improved hydrogel and streptavidin.

FIG. 4: Chromatographic lateral flow Images showing limits of detection of test strips with Strep-CBD immobilized in the nitrocellulose membrane.

FIG. 5 illustrates an exemplary photochemical process to generate a polyacrylamide hydrogel network with protein immobilized in the hydrogel test zone.

FIG. 6: Images showing limits of detection of test strips with capture protein molecules immobilized in the hydrogel test zone formed by different initiators.

FIG. 7: (panel a) Sequences (SEQ ID No. 2: TTTTTTTTTTTTTTTTGTAAAACGACGGCCAGT) of capture DNA immobilized in lateral flow strips and sequence (SEQ ID No. 3: TTTTTTTTTTTTTTTACTGGCCGTCGTTTTACA) of the complementary reporter probe immobilized on gold nanoparticles; (panel b) Chromatographic lateral flow Images showing the capture of gold nanoparticles.

FIG. 8: Image of an acrylamide solution line printed on an 8 cm nitrocellulose membrane.

### SUMMARY

Provided herein, in one aspect, is a system for detecting or identifying an analyte, comprising:
a lateral flow test strip;
a test zone situated on the lateral flow test strip, comprising an affinity molecule immobilized thereon, wherein the affinity molecule is pre-selected to have a binding specifity with an analyte of interest; and
a hydrogel composition deposited on the test zone, comprising an anchor moeity embedded therein, wherein the anchor moiety is pre-selected to bind or react with the affinity molecule, thereby immobilizing the affinity molecule on the test zone;
wherein the hydrogel composition comprises about 0.01% to 20%, or 0.01% to 10%, or about 0.1% to 5%, or about 0.5% to 5%, or about 0.5% to 2% by weight modified monomers, each modified monomer comprising the anchor moiety.

In some embodiments, the lateral flow test strip can include a substrate, a sample pad, a probe conjugate pad, a detection pad, and an absorbent pad, and wherein the detection pad comprises the test zone and a control line.

In some embodiments, on the lateral flow test strip, the sample pad can be positioned at a first end, followed by the probe conjugate pad and the absorbent pad at a second end. In some embodiments, the probe conjugate pad is positioned at a first end, followed by the sample pad and the absorbent pad at a second end.

In some embodiments, the test zone can be shaped to be a line, a square, a circle, an oval, or any combination thereof.

In some embodiments, the analyte of interest can be selected from a nucleic acid, an oligonucleotide, a protein, a peptide, an antibody, an antigen, a carbohydrate, an epitope, a metabolite, a biomarker, and any combination thereof. Accordingly, the affinity molecule can be selected depending on the analyte.

In some embodiments, the hydrogel composition can be made from a polymeric material selected from a polyacrylamide, a poly(ethylene glycol), a polysaccharide, a polypeptide, a copolymer of two or more polymers, and any combination thereof.

In some embodiments, the hydrogel composition can be formed by photoirradiation with a mixture of the modified monomer, monomer, crosslinker, and photoinitiator, wherein the photoinitiator is selected from benzophenone and its derivatives, benzoylphenyl-acrylamides, azo initiators, and any combination thereof.

In some embodiments, the crosslinker can be a derivative of a diacrylate, a bisacrylamide, an ethylene glycol diacrylate, or any combination thereof.

In some embodiments, the monomer can be selected from an acrylamide, an acrylic ester, a water-soluble derivative of acrylic acid, and any combination thereof.

In some embodiments, the monomer can be present in the mixture at a concentration of from about 2% to 30% by weight, preferably 3%-10%.

In some embodiments, a ratio of the monomer to the crosslinker can be about 200:1 to 1:0, preferably about 50:1 to 10:1.

In some embodiments, a ratio of the photoinitiator to the mixture can be about 0.01% to 10%, preferably 0.1% to 1%.

In some embodiments, the anchor moiety comprises a protein such as streptavidin, a receptor protein and other protein capable of binding to a target.

In some embodiments, the modified monomer can include an acrylate derivative bearing a functional group, such as an acrylate amine, an acrylate oxyamine, an acrylate hydrazine, an acrylate boric acid, or any combination thereof.

In some embodiments, the modified monomer can include a methacrylate derivative bearing a functional group, such as a methacrylate amine, a methacrylate oxyamine, a methacrylate hydrazine, a methacrylate boric acid, or any combination thereof.

In some embodiments, the modified monomer can include an acrylamide derivative bearing an oligonucleotide, a peptide, or an oligosaccharide, or any combination thereof.

Also provided herein is a method for detecting or identifying an analyte, comprising:
(a) providing any one of the systems disclosed herein;
(b) flowing a sample through the lateral flow test strip; and
(c) detecting the presence or absence or amount of the analyte of interest.

### DETAILED DESCRIPTION

This disclosure, in one aspect, is related to a lateral flow strip with polymeric hydrogel deposited at one or more test lines and/or control lines for the immobilization of affinity molecules (probes), including but not limited to nucleic acids (for example, DNA or RNA probes), proteins (for example, antibody, antigen, receptor, ligands), and carbohydrates (for instance, glycans and polysaccharides). As a result, the lateral flow strip can be used to detect various analytes depending on the affinity molecule or probe to which the analyte is pre-designed to bind.

This disclosure also provides methods for preparing hydrogel test lines and the immobilization of capture molecules in the test lines. As a result, the said Lateral flow test strip has a lower limit of detection (LoD) and/or higher sensitivity than those conventional ones in which the capture molecules are immobilized by physical adsorption. The increase in detection sensitivity is unexpectedly high, ranging from 10 fold to 200 fold. Such surprising result is, without wishing to be bound by theory, believed to be achieved by a combination of factors, including the improved hydrophilicity of the test lines due to the hydrogel, the trapping of anchor molecules in the hydrogel that can interact with the affinity molecules and immobilize (e.g., permanently if covalently reacted with the anchor moiety) these affinity molecules onto the test line and/or control line, and the controlled orientation of the immobilized affinity molecules that facilitates the capture of target analytes in the test sample, among other things.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

An “analyte” refers to a target molecule to be analyzed or detected by the methods and systems disclosed herein. Generally this is achieved by way of binding to an affinity molecule that has specificity to the analyte.

An “affinity molecule” is used herein to refer to a molecule that is pre-selected or pre-designed to have an affinity against an analyte of interest. Examples include nucleic acids (for example, DNA or RNA probes), proteins (for example, antibody, antigen, receptor, ligands), and carbohydrates (for instance, glycans and polysaccharides).

An “anchor moeity” or “anchor molecule” is used herein to refer to a moeity or molecule that is pre-selected or pre-designed to bind with, react with (e.g., chemical reaction via a functional group) or attract affinity molecules. Anchor moeities can be embedded (e.g., physically trapped or covalently linked) in the hydrogel during polymerization. Examples of anchor moeities include: (1) a protein molecule, such as streptavidin, receptor proteins and other binding proteins; (2) an acrylate derivative bearing a functional group, such as acrylate amine, acrylate oxyamine, acrylate hydrazine, acrylate boric acid, etc.; (3) a methacrylate derivative bearing a functional group, such as methacrylate amine, methacrylate oxyamine, methacrylate hydrazine, methacrylate boric acid, etc.; or (4) an acrylamide derivative bearing an oligonucleotide, a peptide, an oligosaccharide, etc.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The term “antigen” herein is used in the broadest sense and encompasses various molecules or molecular structures, such as may be present on the outside of a pathogen, that can be bound by an antigen-specific antibody or B-cell antigen receptor.

“SARS-Cov-2” refers to a novel coronavirus now called severe acute respiratory syndrome coronavirus 2 (SARS-CoV-2; formerly called 2019-nCoV), including its variants of interest and variants of concern, as defined by the World Health Organization (WHO).

“Covid-19” refers to an infectious disease caused by a novel coronavirus now called severe acute respiratory syndrome coronavirus 2 (SARS-CoV-2; formerly called 2019-nCoV).

By “marker” or “biomarker” is meant any protein or polynucleotide having an alteration in expression level or activity that is associated with a disease or disorder.

“Sample” or “tissue sample” refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bone marrow or any bone marrow constituents; bodily fluids such as urine, cerebral spinal fluid, whole blood, plasma and serum. The sample can include a non-cellular fraction (e.g., urine, plasma, serum, or other non-cellular body fluid). In other embodiments, the body fluid from which the sample is obtained from an individual comprises blood (e.g., whole blood).

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

Unless defined otherwise, all technical and scientific terms used herein have the same meaning as those commonly understood by one of ordinary skill in the art to which this disclosure belongs. Although methods and materials similar or equivalent to those described herein can be used in the practice or testing of the present disclosure, suitable methods and materials are described below. The materials, methods, and examples are illustrative only and are not intended to be limiting. All publications, patents, and other documents mentioned herein are incorporated by reference in their entirety.

### Lateral Flow Strips

A lateral flow strip or platform can include a substrate, a sample pad, a probe conjugate pad, a detection pad, and an absorbent pad, as well as one or more test lines and control lines located on the detection pad. An exemplary lateral flow strip is disclosed in PCT International Application No. PCT/US2021/32809, incorporated herein by reference. The present disclosure provides methods and compositions with improved detection sensitivity as well as improved hydrogel composition. Improved sample processing steps are also provided.

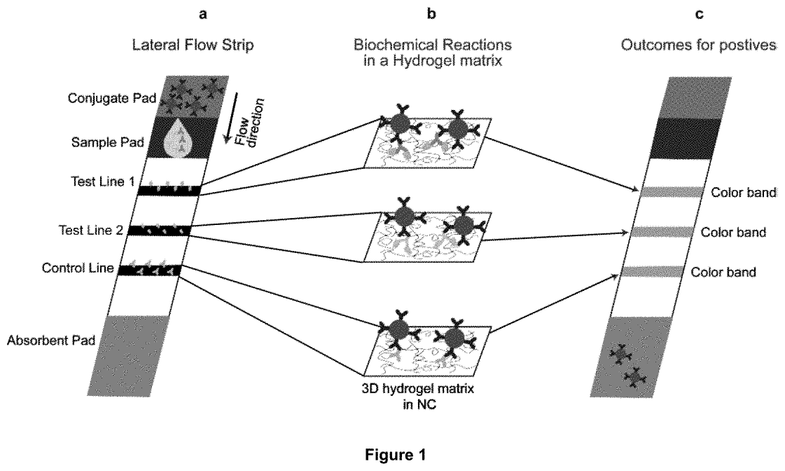

As an example, FIG. 1 illustrates a lateral flow test strip with two antigens (test lines) immobilized in the hydrogel matrixes to detect target antibodies in a blood sample. At one end, the test strip is stuck to a conjugate pad loaded with nanoparticle-antibody conjugates followed by a sample pad, and its other end adheres to an absorbent pad (FIG. 1, panel a). Following the addition of a sample solution to the sample pad, a buffer is engaged in the conjugate pad to flow solutes and conjugates to the detecting lines chromatographically. As shown in FIG. 1, panel b, the antibodies are specifically captured by antigens in the hydrogel test lines and tagged by the nanoparticle-antibody conjugates. As a result, the detection (test) lines are lit up to signal the existence of diagnostic markers (FIG. 1, panel c). As an example, for colorimetric readout, the nanoparticles can be gold or other types of materials, such as carbon, silicon, silver, or nano magnetic beads, and so on, and for fluorescent readout, they can be quantum dots.

In some embodiments, the lateral flow strip can have the sample pad located or positioned at the very end, followed by the probe conjugate pad, the detection pad and the absorbent pad. The sample can be introduced at the sample pad, following the addition of a buffer solution. The buffer solution drives the sample flowing through the substrate towards the absorbent pad by capillary effect. The target molecules (analytes) in the sample are captured by an affinity probe immobilized in a test line so to generate colorimetric signals.

### Hydrogel Test Lines

Hydrogel test lines can be prepared using various polymeric materials such as polyacrylamide, poly(ethylene glycol), polysaccharide, polypeptide, or a copolymer of two or more of the foregoing polymers. The polymer can be naturally occuring or can be synthesized.

In some embodiments, one or more polyacrylamide hydrogel test lines are created on lateral flow strips for detecting pathogens of different biological origins or pathogen related biomarkers. For example, a photolithographic process can be implemented for fabricating the hydrogel lines onto a nicrocellulose (NC) membrane, which uses a photomask to form hydrogel lines (or pads) at predefined locations (see, e.g, PCT International Application No. PCT/US2021/32809, incorporated herein by reference in its entirety).

In some embodiments, the hydrogel lines can be printed or spotted onto the NC membrane without the use of a photomask. In principle, a polyacrylamide (PAA) hydrogel can be formed under photo-irradiation in the presence of an initiator.⁷ In PCT/US2021/32809, bisacrylamide is mixed with polyacrylamide to make hydrogel by either photoinitiation with a photoinitiator or chemical initiation with a chemical initiator. The hydrogel patterned lines may comprise an NHS ester or streptavidin or a fusion protein. A hydrazine is used as the photoinitiator. In this disclosure, in addition to polyacrylamide and bisacrylamide, poly(ethylene glycol) (PEG), polysaccharides, polypeptides and other co-polymers are used for hydrogel materials. Monomers for hydrogel formation include all acrylates besides polyacrylamide, such as acrylic esters and water soluble derivatives of acrylic acid, etc. Monomer solution also contains additional anchor moeities that can be physically or chemically trapped in the hydrogel and interact with the affinity molecules to facilitate immobilization of the affinity molecules. Examples of anchor moeities include: (1) a protein molecule, such as streptavidin, receptor proteins and other binding proteins; (2) an acrylate derivative bearing a functional group, such as acrylate amine, acrylate oxyamine, acrylate hydrazine, acrylate boric acid, etc.; (3) a methacrylate derivative bearing a functional group, such as methacrylate amine, methacrylate oxyamine, methacrylate hydrazine, methacrylate boric acid, etc.; or (4) an acrylamide derivative bearing an oligonucleotide, a peptide, an oligosaccharide, etc. Additional crosslinkers besides bisacrylamide include all derivatives of diacrylate, ethylene glycol diacrylate, etc. The photoinitiator further includes benzophenone and its derivatives, benzoylphenyl-acrylamides, as well as azo initiators that can form radicals under photoirradiation.

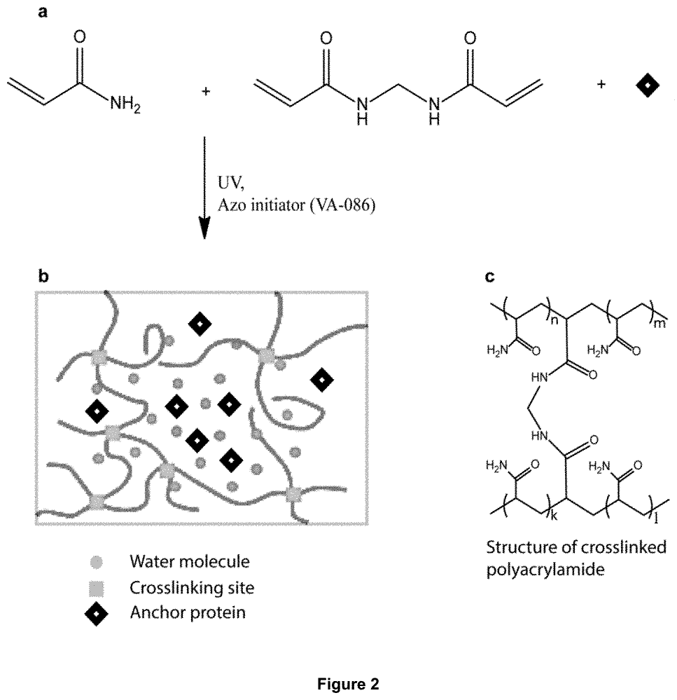

As an example, a solution of acrylamide and bis-acrylamide mixed with protein was dispensed on the nitrocellulose membrane, irradiated with UV light in the presence of an azo-initiator (FIG. 2, panel a). As a result, a polyacrylamide hydrogel network (FIG. 2, panel b) is formed and tangled in the membrane with a chemical structure shown in FIG. 2, panel c. Since the azo initiator decomposes into carbon radicals, which initiate the polymerization, the protein molecules are immobilized in the hydrogel by trapping. In a typical case of forming hydrogel, an acrylamide solution (3.5 μL) comprising 5% acrylamide and bis-acrylamide (39:1) with an azo-initiator (0.1% VA-086) plus 0.13% of streptavidin is dispensed on nitrocellulose into a 1 mm×3 cm line, followed by UV irradiation at 365 nm for 3 minutes to form a hydrogel test zone.

Generally, a concentration of 2% to 30% by weight, preferably 3%-10% for the acrylamide and bis-acrylamide mixture in solution with acrylamide and bis-acrylamide mixing ratio 200:1 to 1:0, preferably 50:1 to 10:1, can be used depending on the type of the pathogen to be tested, the membrane, and the conjugation chemistry. The ratio of photoinitiator to the acrylamide and bis-acrylamide mixture is usually about 0.01% to 10% by weight, preferably 0.1% to 1%. The solution concentration of the anchor molecule, preferably acrylate or methacrylate or acrylamide derivatives, to the acrylamide and bis-acrylamide mixture can be about 0.01% to 10% by weight, preferably 0.1% to 5%. Usually, when the anchor molecule is a protein, its concentration is to the lower end (e.g., about 0.01% to 5% or about 0.01% to 1%), while when it is a derivative of acrylate or methacrylate or acrylamide, its concentration is to the higher end (e.g., about 0.1% to 10% or about 0.5% to 5%) due to its better compatibility with the hydrogel monomer.

In some embodiments, the PAA hydrogel lines are prepared using benzophenone as a photoinitiator. Benzophenone (BP) is known as a type II Norrish photoinitiator. When irradiated by UV-light, it is elevated to an excited state, which can abstract a hydrogen atom from a hydrogen donor to generate a free radical for

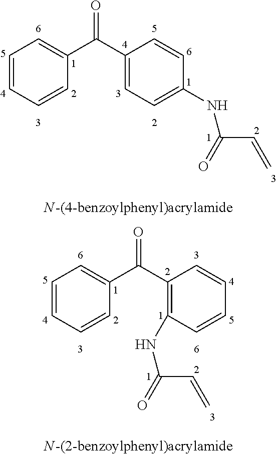 [SMILES: C=CC(=O)Nc1ccc(C(=O)c2ccccc2)cc1 ; C=CC(=O)Nc1ccccc1C(=O)c1ccccc1]

polymerization.⁹ The H-donors can be amine, alcohol, thiol, or other moieties. Also, BP abstracts hydrogen from proteins and polysaccharides.^{10, 11} The present disclosure provides a method to covalently immobilize antibodies in nitrocellulose membranes.

The photoinitiator is polymerizable in some embodiments, which has a structure below, but not limited to them.

In some embodiments, the acrylamide solution contains one of the following monomers or their combinations for the polymerization. Each of these monomers bears a function group for attaching biomolecules and probe molecules.

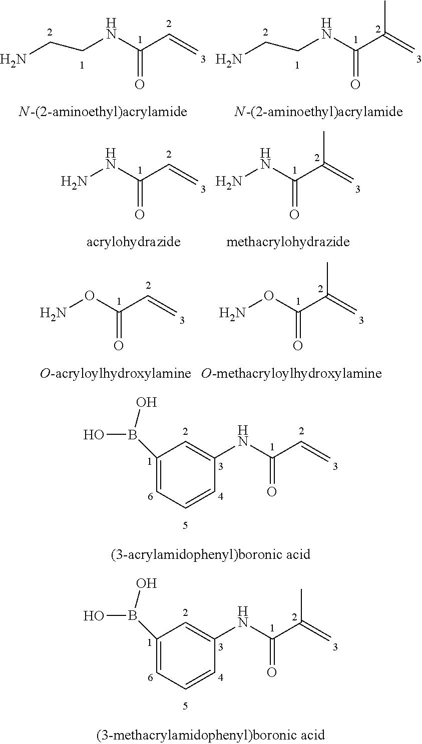

In some embodiments, the acrylamide solution contains oligonucleotide acrydites with a general structure shown below. The oligonucleotide comprises natural and artificial constituents.

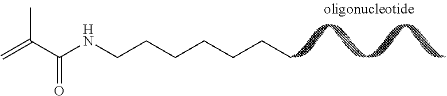

In some embodiments, the hydrogel monomer has one of the chemical structures, as shown below.

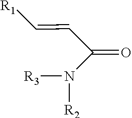

R₁═CH₃
R₂, R₃═H, CH₃, CH(CH₃)₂, oligo(ehtylene glycol), peptides, oligosaccharides

In some embodiments, the hydrogel monomer is an acrylic ester with one of the chemical structures, as shown below.

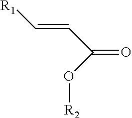

R₁═H, CH₃
R₂, ═H, CH₃, CH(CH₃)₂, oligo(ehtylene glycol), peptides, oligosaccharides

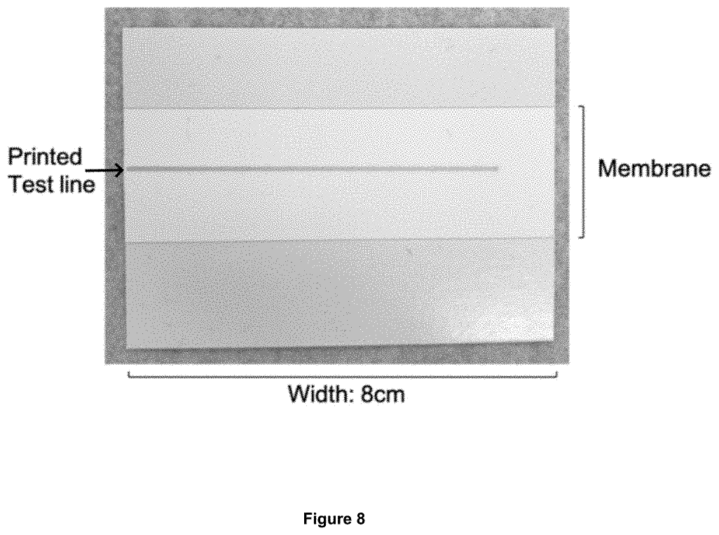

In some embodiments, the monomer solution can be printed on a lateral flow test strip using an inkjet printer in the shape of a line, as shown in FIG. 8.

In some embodiments, the monomer solution can be printed on the substrate in a circle or oval shape, similar to a microarray. This way, more analytes can be detected in one assay. The circles or ovals can be arranged in various configurations.

### Diagnostic Uses

In various embodiments, the lateral flow strips and methods disclosed herein can be used to detect the presence or absence of one or more target analytes such as nucleic acid sequences (e.g., a nucleic acid sequence of one or more pathogens), antigens (e.g., an antigen of one or more pathogens), antibodies (e.g., an antibody elicited by a vaccine against a pathogen). In some embodiments, the pathogens are viral, bacterial, fungal, parasitic, or protozoan pathogens, such as SARS-CoV-2 or an influenza virus.

Target nucleic acid sequences, antigens and/or antibodies may be associated with a variety of diseases or disorders, as described below. In some embodiments, the lateral flow strips and methods are used to diagnose at least one disease or disorder caused by a pathogen. In certain instances, the lateral flow strips and methods are configured to detect a nucleic acid encoding a protein (e.g., a nucleocapsid protein) of SARS-CoV-2, or an antigen of SARS-CoV-2, or an antibody elicited by a SARS-CoV-2 vaccine. In some embodiments, the lateral flow strips and methods are configured to identify particular strains of a pathogen (e.g., a virus).

In some embodiments, the lateral flow strips and methods are configured to detect a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In some embodiments, the viral pathogen is SARS-CoV-2. In some embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

The lateral flow platform (or test strip) disclosed herein can be used for detecting not only viral or bacterial pathogens, but also for other types of pathogens or pathogen-related biomarkers or biomolecules, including but not limited to antibodies, antigens, epitopes, DNA, RNA, and metabolites.

### EXAMPLES

### Example 1

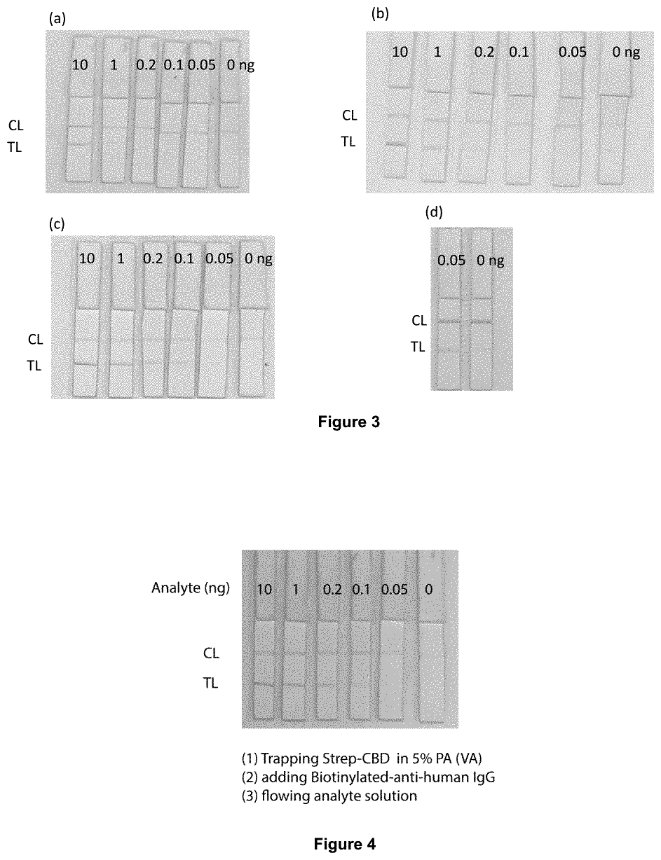

The lateral flow strip prepared in accordance with methods of the present disclosure was tested for its sensitivity in detecting an anti-S1 fragment of SARS-CoV-2's spike protein antibody using a dipstick (half-strip) assay.⁸ First, gold nanoparticles with a 50 nm diameter were coated with the S1 protein for capturing the anti-S1 antibodies, and biotin-SP-conjugated anti-human IgG Fc was used as a secondary antibody to capture the anti-S1 antibodies in the test line (TL). This experiment demonstrates how the way to immobilize an antibody affects the sensitivity of detection. In FIG. 3, panel a, biotin-SP-conjugated anti-human IgG Fc was immobilized in the test line by conventional physical adsorption. It shows that the strip has a limit of detection of 10 ng. In FIG. 3, panel b, biotin-SP-conjugated anti-human IgG Fc was immobilized in the test line by first adding it into a 5% acrylamide solution and then dispensed at the test line, followed by photoirradiation. This test strip detected the anti-S1 antibody as low as 1.0 ng. Compared to the physical adsorption, this treatment has improved the sensitivity by tenfold. The sensitivity was further enhanced by immobilizing the secondary antibody through the streptavidin-biotin interactions. In the same manner, the streptavidin was immobilized in the test line by being added to mix with the 5% acrylamide and then dispensed at the test line, followed by photoirradiation. Then, the biotin-SP-conjugated anti-human IgG Fc antibody was added to the test line and incubated at room temperature for about one hour. FIG. 3, panel c shows the test strip has detected the anti-S1 antibody down to 0.1 ng. A separate experiment shows that the sensitivity can achieve a limit of detection as low as 0.05 ng of anti-S1 antibody (FIG. 3, panel d), a surprising 200-fold improvement.

In one embodiment, the streptavidin is a streptavidin-carbohydrate anchor module (Strep-CBD) recombinant protein. The following is the sequence of Streptavidin-CBD with hexahistidine tag on N-terminus.

SEQ ID No. 1:
MGHHHHHHSHASMTGGQQMGRDEAGITGTWYNQLGSTFIVTAGADGAL
TGTYESAVGNAESRYVLTGRYDSAPATDGSGTALGWTVAWKNNYRNAH
SATTWSGQYVGGAEARINTQWLLTSGTTEANAWKSTLVGHDTFTKVKP
SAASIDAAKKAGVNNGNPLDAVQQTGNSGLTTNPGVSAWQVNTAYTAG
QLVTYNGKTYKCLQPHTSLAGWEPSNVPALWQLQ

The Strep-CBD can be trapped in the PAA test line in the same way as described above. The Strep-CBD lateral flow strip shows a similar sensitivity to those with streptavidin in the PAA test line (FIG. 4).

### Example 2

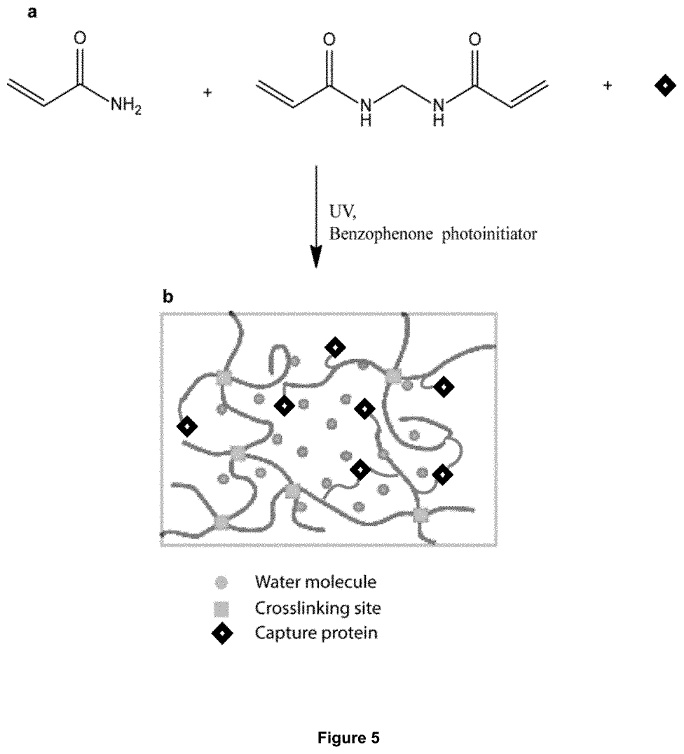

Lateral flow strips containing PAA hydrogel lines are prepared using benzophenone (BP) as a phtoinitiator as disclosure herein. Specifically, in the presence of 0.1% BP, a 5% acrylamide solution containing 0.1% biotin-conjugated anti-human IgG Fc was dispensed in nitrocellulose and irradiated by UV-light at 365 nm for 3 minutes to form a hydrogel test zone. As shown in FIG. 5, the antibody molecules were covalently attached to the PAA hydrogel and the membrane.

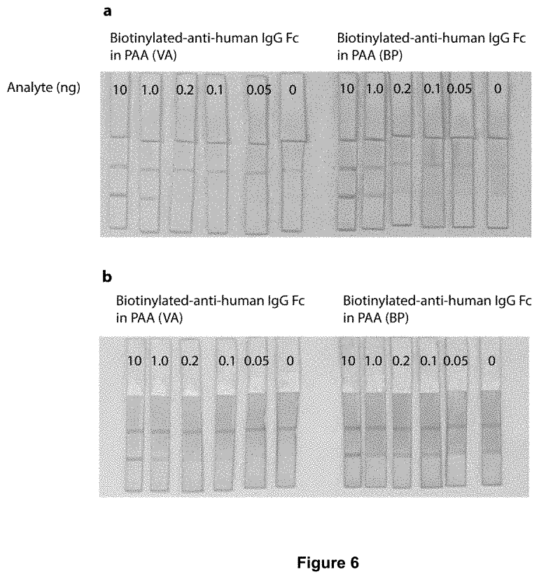

Such lateral flow strips can detect the antibody analyte with a similar sensitivity compared to those immobilized in the hydrogel generated by VA (FIG. 6, panel a). A post-assay treatment with a protein stain ponceau indicates that the chemically immobilized capture proteins were well remained in the test zone compared to those physically trapped that leached out (FIG. 6, panel b).

### Example 3

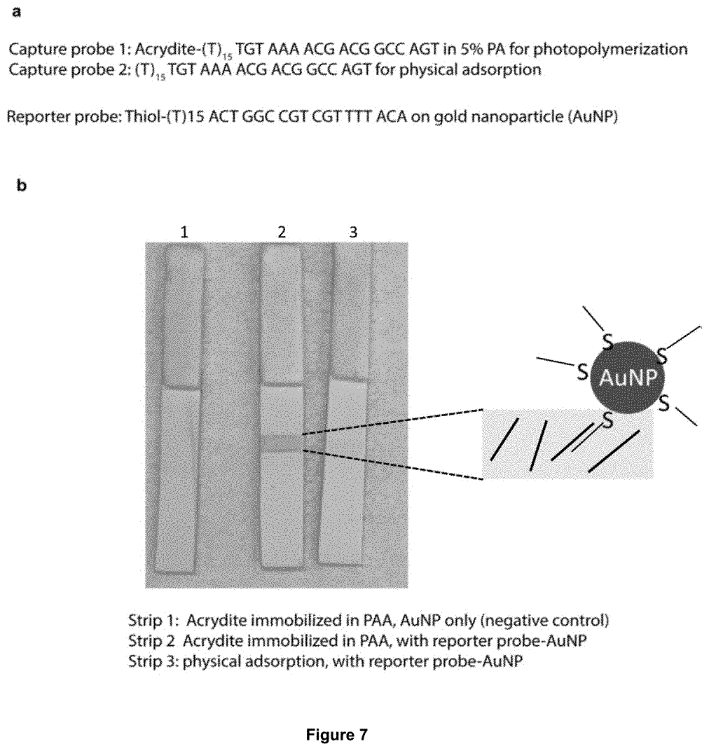

In one embodiment, an oligonucleotide acrydite was incorporated into the PAA hydrogel test line as a capture probe by the photopolymerization as described above, and another oligonucleotide with the same sequence was directly spotted on the nitrocellulose membrane. The complementary reporter oligonucleotide was attached to gold nanoparticles for testing. The sequences of these oligonucleotides are listed in FIG. 7, panel a. With naked gold nanoparticles flowing through Strip 1 (FIG. 7, panel b), there was no color appearing in the PAA test line, where the capture probe was covalently attached. In the same way, flowing the gold nanoparticles conjugated with the reporter probe produced the color in the PAA test line, indicating that the reporter probe was hybridized to the capture probe because they complement each other (Strip 2, FIG. 7, panel b). In comparison, the capture probe immobilized by physical adsorption only created a faint color (Strip 3, FIG. 7, panel b).

### Modifications

While a description of various embodiments has illustrated the present disclosure and while these embodiments have been described in considerable detail, it is not the intention of the applicants to restrict or in any way limit the scope of the appended claims to such detail.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

1. Kissler, S. M.; Tedijanto, C.; Goldstein, E.; Grad, Y. H.; Lipsitch, M., Projecting the transmission dynamics of SARS-CoV-2 through the postpandemic period. Science 2020, 368 (6493), 860-868.
2. Service, R. F., Fast, cheap tests could enable safer reopening. Science 2020, 369 (6504), 608-609.
3. Lippi, G.; Plebani, M., SARS-CoV-2 antibodies titration: a reappraisal. Annals of Translational Medicine 2020, 8 (16), 1032.
4. Egia-Mendikute, L.; Bosch, A.; Prieto-Fernandez, E.; Lee, S. Y.; Jimenez-Lasheras, B.; Garcia del Rio, A.; Antonana-Vildosola, A.; Bruzzone, C.; Bizkarguenaga, M.; Embade, N.; Abrescia, N. G. A.; Mato, J. M.; Millet, O.; Palazon, A., Sensitive detection of SARS-CoV-2 seroconversion by flow cytometry reveals the presence of nucleoprotein-reactive antibodies in Covid-19-naive individuals. medRxiv 2020, 2020.07.28.20162941.
5. Li, Z.; Yi, Y.; Luo, X.; Xiong, N.; Liu, Y.; Li, S.; Sun, R.; Wang, Y.; Hu, B.; Chen, W.; Zhang, Y.; Wang, J.; Huang, B.; Lin, Y.; Yang, J.; Cai, W.; Wang, X.; Cheng, J.; Chen, Z.; Sun, K.; Pan, W.; Zhan, Z.; Chen, L.; Ye, F., Development and Clinical Application of A Rapid IgM-IgG Combined Antibodfile:///C:/Users/pzhan/Downloads/jmv.25727.pdfy Test for SARS-CoV-2 Infection Diagnosis. Journal of Medical Virology n/a (n/a).
6. Lassaunière, R.; Frische, A.; Harboe, Z. B.; Nielsen, A. C. Y.; Fomsgaard, A.; Krogfelt, K. A.; Jørgensen, C. S., Evaluation of nine commercial SARS-CoV-2 immunoassays medRxiv 2020.
7. Yang, C.; Yin, T.; Suo, Z., Polyacrylamide hydrogels. I. Network imperfection. Journal of the Mechanics and Physics of Solids 2019, 131, 43-55.
8. Grant, B. D.; Anderson, C. E.; Williford, J. R.; Alonzo, L. F.; Glukhova, V. A.; Boyle, D. S.; Weigl, B. H.; Nichols, K. P., SARS-CoV-2 Coronavirus Nucleocapsid Antigen-Detecting Half-Strip Lateral Flow Assay Toward the Development of Point of Care Tests Using Commercially Available Reagents. Analytical Chemistry 2020, 92 (16), 11305-11309.
9. Dadashi-Silab, S.; Doran, S.; Yagci, Y., Photoinduced Electron Transfer Reactions for Macromolecular Syntheses. Chem Rev 2016.
10. Wu, X.; Tang, Q.; Liu, C.; Li, Q.; Guo, Y.; Yang, Y.; Lv, X.; Geng, L.; Deng, Y., Protein photoimmobilizations on the surface of quartz glass simply mediated by benzophenone. Applied Surface Science 2011, 257 (17), 7415-7421.
11. Ritz, U.; Kögler, P.; Höfer, I.; Frank, P.; Klees, S.; Gebhard, S.; Brendel, C.; Kaufmann, K.; Hofmann, A.; Rommens, P. M.; Jonas, U., Photocrosslinkable polysaccharide hydrogel composites based on dextran or pullulan-amylose blends with cytokines for a human co-culture model of human osteoblasts and endothelial cells. Journal of Materials Chemistry B 2016, 4 (40), 6552-6564.

1. A system for detecting or identifying an analyte, comprising:
a lateral flow test strip,
a test zone situated on the lateral flow test strip, comprising an affinity molecule immobilized thereon, wherein the affinity molecule is pre-selected to have a binding specifity with an analyte of interest; and
a hydrogel composition deposited on the test zone, comprising an anchor moeity embedded therein, wherein the anchor moiety is pre-selected to bind or react with the affinity molecule, thereby immobilizing the affinity molecule on the test zone;
wherein the hydrogel composition comprises about 0.01% to 20%, or 0.01% to 10%, or about 0.1% to 5%, or about 0.5% to 5%, or about 0.5% to 2% by weight modified monomers, each modified monomer comprising the anchor moiety.

2. The system of claim 1, wherein the lateral flow test strip comprises a substrate, a sample pad, a probe conjugate pad, a detection pad, and an absorbent pad, and wherein the detection pad comprises the test zone and a control line.

3. The system of claim 2, wherein on the lateral flow test strip, the sample pad is positioned at a first end, followed by the probe conjugate pad and the absorbent pad at a second end.

4. The system of claim 2, wherein on the lateral flow test strip, the probe conjugate pad is positioned at a first end, followed by the sample pad and the absorbent pad at a second end.

5. The system of claim 1, wherein the test zone is shaped to be a line, a square, a circle, an oval, or any combination thereof.

6. The system of claim 1, wherein the analyte of interest is selected from a nucleic acid, an oligonucleotide, a protein, a peptide, an antibody, an antigen, a carbohydrate, an epitope, a metabolite, a biomarker, and any combination thereof.

7. The system of claim 1, wherein the hydrogel composition comprises a polymeric material selected from a polyacrylamide, a poly(ethylene glycol), a polysaccharide, a polypeptide, a copolymer of two or more polymers, and any combination thereof.

8. The system of claim 1, wherein the hydrogel composition is formed by photoirradiation with a mixture of the modified monomer, monomer, crosslinker, and photoinitiator, wherein the photoinitiator is selected from benzophenone and its derivatives, benzoylphenyl-acrylamides, azo initiators, and any combination thereof.

9. The system of claim 8, wherein the crosslinker is a derivative of a diacrylate, a bisacrylamide, an ethylene glycol diacrylate, or any combination thereof.

10. The system of claim 8, wherein the monomer is selected from an acrylamide, an acrylic ester, a water-soluble derivative of acrylic acid, and any combination thereof.

11. The system of claim 8, wherein the monomer is present in the mixture at a concentration of from about 2% to 30% by weight, preferably 3%-10%.

12. The system of claim 8, wherein a ratio of the monomer to the crosslinker is 200:1 to 1:0, preferably 50:1 to 10:1.

13. The system of claim 8, wherein a ratio of the photoinitiator to the mixture is about 0.01% to 10%, preferably 0.1% to 1%.

14. The system of claim 1, wherein the anchor moiety comprises a protein.

15. The system of claim 1, wherein the modified monomer comprises an acrylate derivative bearing a functional group, such as an acrylate amine, an acrylate oxyamine, an acrylate hydrazine, an acrylate boric acid, or any combination thereof.

16. The system of claim 1, wherein the modified monomer comprises a methacrylate derivative bearing a functional group, such as a methacrylate amine, a methacrylate oxyamine, a methacrylate hydrazine, a methacrylate boric acid, or any combination thereof.

17. The system of claim 1, wherein the modified monomer comprises an acrylamide derivative bearing an oligonucleotide, a peptide, or an oligosaccharide, or any combination thereof.

18. A method for detecting or identifying an analyte, comprising:
providing the system of claim 1;
flowing a sample through the lateral flow test strip; and
detecting the presence or absence or amount of the analyte of interest.


SEQUENCE LISTING (3 sequences)

SEQ ID NO: 1 - protein, 226 aa, unidentified
  REGION 1..226: note = Synthetic
MGHHHHHHSHASMTGGQQMGRDEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQTGNSGLTTNPGVSAWQVNTAYTAGQLVTYNGKTYKCLQPHTSLAGWEPSNVPALWQLQ

SEQ ID NO: 2 - DNA, 33 bp, unidentified
  misc_feature 1..33: note = Synthetic
  source 1..33: mol_type = other DNA
ttttttttttttttttgtaaaacgacggccagt

SEQ ID NO: 3 - DNA, 33 bp, unidentified
  misc_feature 1..33: note = Synthetic
  source 1..33: mol_type = other DNA
tttttttttttttttactggccgtcgttttaca